(19) United States
(12) Patent Application Publication
(10) Pub. No.: US 2006/0099254 A1
(43) Pub. Date: May 11, 2006

(54) Sustained release drug delivery system and method

(76) Inventors: Divyakant S. Desai, West Windsor, NJ (US); Sanjeev H. Kothari, Princeton, NJ (US); Laxmikant H. Sharma, Indianapolis, IN (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/266,516

(22) Filed: Nov. 2, 2005

Related U.S. Application Data
(60) Provisional application No. 60/624,388, filed on Nov. 2, 2004.

(51) Int. Cl. A61K 31/522; A61K 9/22; A61K 31/16; A61K 31/175

(52) U.S. Cl. 424/468; 514/263.34; 514/592; 514/629

(57) ABSTRACT

The present invention relates to a pharmaceutical delivery system comprising a gel-like structure that comprises at least one water-soluble polymer, such as, for example, povidone or hydroxypropyl cellulose, and at least one fatty acid, such as, for example, stearic acid or lauric acid. The invention further relates to a sustained release drug delivery composition comprising the gel-like structure and at least one drug trapped or dissolved therein, wherein said system is capable of releasing the drug in a dissolution medium at a controlled rate. The invention is also directed to a method for preparing the sustained release drug delivery composition.

### RELATED APPLICATION

This application claims priority benefit under Title 35 §119(e) of U.S. Provisional Application No. 60/624,388, filed Nov. 2, 2004, the contents of which are herein incorporated by reference in its entirety.

### FIELD OF THE INVENTION

The present invention relates to a pharmaceutical delivery system comprising a gel-like structure that comprises a water-soluble polymer, such as, for example, povidone or hydroxypropyl cellulose, and a fatty acid, such as, for example, stearic acid or lauric acid. The invention further relates to a sustained release drug delivery composition comprising the gel-like structure and a drug trapped or dissolved therein, wherein said system is capable of releasing the drug in a dissolution medium at a controlled rate. The invention is also directed to a method for preparing the sustained release drug delivery composition.

### BACKGROUND OF THE INVENTION

A sustained release or controlled release drug delivery system can be useful in enhancing patient compliance by reducing the frequency with which medicines need to be administered. A variety of approaches have been used in the art to produce sustained or controlled release drug delivery systems. Such approaches include, for example, either coating a tablet or bead with polymeric material, or making a tablet with insoluble or poorly soluble polymers. Each of these approaches, however, has drawbacks.

The coating of a tablet or bead, for example, is time consuming, and, as an aqueous coating is usually employed, in general cannot be used when the drug contained in such tablet or bead is moisture sensitive. Likewise, the lot to lot variability of polymers may cause tablets produced solely with insoluble or poorly soluble polymers to exhibit unreproducible performance profiles.

Accordingly, the sustained release drug delivery system described herein is designed to address the drawbacks associated with such prior art approaches.

### SUMMARY OF THE INVENTION

The present invention relates to a pharmaceutical delivery composition capable of delivering a drug, such as, for example, theophylline, glipizide or acetaminophen, in a sustained or controlled release manner, wherein such composition is in the form of a gel-like structure comprising:

a) at least one water-soluble polymer; and

b) at least one fatty acid.

The present invention further relates to a sustained release drug delivery composition capable of delivering a drug, such as, for example, theophylline, glipizide or acetaminophen, in a sustained or controlled release manner, wherein such composition comprises:

a) at least one drug; and

b) a gel-like structure comprising:
1) at least one water-soluble polymer; and
2) at least one fatty acid,
   wherein the drug is trapped or dissolved in the gel-like structure and can be released from such structure in a dissolution medium, such as, for example, gastric acid at a sustained or controlled release rate.

The present invention is also directed to a method for preparing a sustained release drug delivery composition comprising:

a) mixing at least one drug, at least one water-soluble polymer, and at least one fatty acid together; and

b) heating the mixture of a) at a sufficiently high temperature to melt the fatty acid and form a solid solution of water-soluble polymer and fatty acid, wherein the drug is either being trapped or dissolved in the solid solution.

The present invention is further directed to a method for preparing a sustained release drug delivery composition comprising:

a) screening each of at least one drug, at least one water soluble polymer, and at least one fatty acid so that the drug, the water-soluble polymer, and the fatty acid each have an average particle size of from about 20 to about 100 microns;

b) mixing the drug, the water-soluble polymer, and the fatty acid together;

c) transferring the mixture of b) to a capsule or a lozenge mold; and

d) heating the mixture of c) to a temperature ranging from about 35° C. to about 55° C. for a period of time ranging from about 0.25 to about 10 hours.

### DETAILED DESCRIPTION OF THE INVENTION

The features and advantages of the present invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

Unless specifically stated otherwise herein, references made in the singular may also include the plural. For example, “a” and “an” may refer to either one, or one or more.

All numbers expressing quantities of ingredients; properties, such as, for example, molecular weight, and reaction conditions; and the like that are preceded by the word “about” are to be understood as only approximations so that slight variations above and below the stated number may be used to achieve substantially the same results as the stated number. Accordingly, unless indicated to the contrary, numerical parameters preceded by the word “about” are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Each of the stated rages are also to be understood as being continuous so as to include each numerical parameter between the stated minimum and maximum value of each range. It is to be further understood that, while not intending to limit the applicability of the doctrine of equivalents to the scope of the claims, each numerical parameter should at least be construed in a manner consistent with the reported number of significant digits for each numerical parameter and by applying ordinary rounding techniques. It is also to be understood that, while not intending to limit the applicability of the doctrine of equivalents to the scope of the claims, even though a number may be contained within a numerical range wherein at least one of the minimum and maximum numbers of the range is or is not preceded by the word “about”, each numerical value contained within the range may or may not be preceded by the word “about”. For Example, a range of about 1 to about 10 includes about 1, about 2, 2, about 3, 3, about 4, 4, about 5, 5, about 6, 6, about 7, 7, about 8, 8, about 9, 9, and about 10; a range of about 1.1 to about 3.2 includes about 1.1, about 1.2, 1.2, about 1.3, 1.3, about 1.4, 1.4, about 1.5, 1.5, about 1.6, 1.6, about 1.7, 1.7, about 1.8, 1.8, about 1.9, 1.9, about 2.0, 2.0, about 2.1, 2.1, about 2.2, 2.2, about 2.3, 2.3, about 2.4, 2.4, about 2.5, 2.5, about 2.6, 2.6, about 2.7, 2.7, about 2.8, 2.8, about 2.9, 2.9, about 3.0, 3.0, about 3.1, 3.1, and about 3.2; and a range of about 1 to 4 includes about 1, 2, about 2, 3, about 3, and 4.

Further, when an amount, concentration, or other value or parameter is given as a list of upper values and lower values, such listings are intended to include all ranges formed by pairing any upper value with any lower value, regardless of whether ranges are separately disclosed.

The definitions set forth herein take precedence over definitions set forth in any patent, patent application, and/or patent application publication incorporated herein by reference.

The term “gel-like structure” as used herein refers to the matrix or lattice formed from heating a mixture of water-soluble polymer and fatty acid, in which matrix a drug may be trapped or dissolved.

The term “water-soluble polymer” as used herein refers to a polymer having a solubility in water at 25° C. ranging from about 200 to about 500 g/L and a molecular weight ranging from about 8,000 to about 1,250,000.

The term “fatty acid” as used herein refers to a saturated or unsaturated carboxylic acid that is either derived from, or contained in an animal, a vegetable fat, or an oil, and which is composed of a chain of alkyl groups containing from 4 to 22 carbon atoms and a terminal carboxyl group.

The present invention is directed to a pharmaceutical delivery composition in the form of a gel-like structure comprised of at least one water-soluble polymer and at least one fatty acid, whereby such composition is designed to deliver a drug trapped or dissolved therein in a sustained release manner. The trapped or dissolved drug particles can be released at a controlled rate when the composition is exposed to a dissolution medium, such as, for example, gastric acid and/or intestinal fluid.

The water-soluble polymer and fatty acid used in accordance with the present invention are generally chosen based on the target release profile of the drug to be delivered, as well as the drug release mechanism to be used, i.e. diffusion controlled versus erosion controlled.

A water-soluble polymer suitable for use herein includes a polymer capable of lowering the melting point of the selected fatty acid, and inhibiting crystallization of the melted fatty acid as it cools. When, for example, stearic acid is selected as the fatty acid, the melting point of such acid is lowered from about 66-69° C. to about 45-50° C.

Examples of water-soluble polymers suitable for use herein include, but are not limited to, for example, povidone; hydroxypropyl cellulose; hydroxypropyl methylcellulose; and polyethylene glycol. In one embodiment, the water soluble polymer is selected from povidone (molecular weight ranging from about 5,000 to about 1,000,000 preferably from about 30,000 to about 1,000,000) and hydroxypropyl cellulose.

Generally, a delivery composition in accordance with the present invention contains from about 20 to about 50%, by weight of the composition, water-soluble polymer. In one embodiment, the delivery composition of the invention contains from about 20 to about 40%, by weight of the composition, water-soluble polymer. In another embodiment, the delivery composition of the invention contains from about 30 to about 35%, by weight of the composition, water-soluble polymer.

The fatty acid suitable for use herein generally melts in the presence of the selected water-soluble polymer at a temperature ranging from about 35 to about 55° C.

Generally, a delivery composition in accordance with the present invention contains from about 20 to about 50%, by weight composition, fatty acid. In one embodiment, the delivery composition of the invention contains from about 20 to about 45%, by weight composition, fatty acid. In another embodiment, the delivery composition of the invention contains from about 30 to about 40% by weight composition, fatty acid.

Examples of fatty acids suitable for use herein include, but are not limited to, for example, saturated fatty acids, such as, for example, lauric acid, palmitic acid, and stearic acid; and unsaturated fatty acids, such as, for example, oleic acid and linoleic acid. In one embodiment, the fatty acid is selected from stearic acid, lauric acid, and palmitic acid.

In general, the weight ratio of water-soluble polymer to fatty acid ranges from bout 2.5:1 to about 1:2. In one embodiment, the weight ratio of water-soluble polymer to fatty acid ranges from about 2:1 to about 1:1.5. In yet another embodiment, the weight ratio of water-soluble polymer to fatty acid is about 1:1.

In one embodiment, the water-soluble polymer is selected from povidone or hydroxypropyl cellulose and the fatty acid is selected from stearic acid or lauric acid.

The water soluble polymer and fatty acid can form a solid solution, wherein the polymer and acid are first mixed together and then exposed to a temperature ranging from about 30° C. to about 60° C.

In one embodiment, each of the polymer and the acid components being mixed together has an average particle size of from about 10 to about 100 microns. That is, each of the polymer and the acid are, prior to being mixed together, separately screened to ensure that prior to being mixed together each component has an average particle size of from about 20 to about 100 microns. In another embodiment, the average particle size of each component ranges from about 30 to about 60 microns.

The present invention is further directed to a sustained release drug delivery composition comprising the gel-like structure described hereinabove and at least one drug, wherein the drug is trapped or dissolved in the gel-like structure and released from the gel-like structure in dissolution medium, such as, for example, gastric acid and/or intestinal fluid at a sustained release rate.

The drug utilized in accordance with the present invention may include any pharmacologically active substance. Exemplary drugs include, but are not limited to, for example, bronchodialators, such as, for example, theophylline and pseudoephedrine; anti-diabetics, such as, for example, glipizide; analgesics, such as, for example, acetaminophen, ibuprofen, naprosyn, morphine sulphate, oxycodone, hydromorphone, fentanyl, and codeine; antipsycotics, such as, for example, aripiprazole; anti-hypertensives, such as, for example, diltiazem, verapamil, nifedipine, and beta-blockers; and anticonvulsants, such as, for example, phenytoin, and divaloprex sodium.

In one embodiment, the pharmacologically active substance is selected from theophylline, acetaminophen, and glipizide.

In another embodiment, the pharmacologically active substance is selected from theophylline, acetaminophen, and glipizide; the water-soluble polymer is selected from povidone and hydroxypropyl cellulose; the fatty acid is selected from stearic acid, palmitic acid, and lauric acid; and the weight ratio of water-soluble polymer to fatty is about 1:1.

The amount of drug contained in the delivery composition of the present invention generally depends on the drug employed, the drug dosage, and the drug release rate needed. In one embodiment, the delivery composition of the present invention contains from about 3 to about 60%, by weight of the composition, drug. In another embodiment, the delivery composition of the present invention contains from about 4 to about 40%, by weight of the composition, drug.

While not wishing to be bound by any particular theory, it is believed that preventing the melted fatty acid from crystallizing as the fatty acid cools creates an amorphous phase that traps the drug in predominately its original crystalline state, unless, that is, the amorphous drug is added at the start, or the drug has a high solubility in the water soluble polymer-fatty acid solid solution. If the drug has a high solubility in the solid solution, a three component (drug, water soluble polymer, and fatty acid) solid solution is formed.

The factors believed to control the rate at which drug is released from the delivery composition of the invention include, but are not limited to, for example, drug loading; particular water-soluble polymer and fatty acid combination chosen; molecular weight and water solubility of the polymer; and particle size of the drug. For example, as the drug load increases there is a proportional increase in the release rate of the drug.

The delivery composition of the present invention may take the form of a capsule, a lozenge, a tablet or other conventional solid dosage form. When in the form of a lozenge, a sweetener, such as, for example, corn syrup, or an artificial sweetener, such as, for example, saccharin and may be present.

In one embodiment, the drug, water-soluble polymer, and fatty acid are mixed together, and then exposed to a temperature of from about 30° C. to 60° C., wherein the water-soluble polymer and fatty acid form the solid solution in which the drug is trapped. In another embodiment, the drug, water-soluble polymer, and fatty acid mixture are exposed to a temperature of from about 35° C. to about 55° C. In yet another embodiment, the drug, water-soluble polymer, and fatty acid mixture are exposed to a temperature of from about 40° C. to about 50° C.

In another embodiment, the drug, water soluble polymer, and fatty acid form a solid solution upon being exposed to a temperature of from about 30° C. to 60° C. In another, the drug, water soluble polymer, and fatty acid form a solid solution upon being exposed to a temperature of from about 35° C. to about 55° C. In yet another embodiment, the drug, water-soluble polymer, and fatty acid mixture form a solid solution upon being exposed to a temperature of from about 40° C. to about 50° C.

In one embodiment, each of the drug, the polymer, and the acid components that are mixed together have an average particle size of from about 20 to about 100 microns. That is, each of the drug, the polymer, and the acid are, prior to being mixed together, separately screened to ensure that prior to being mixed together each component has an average particle size of from about 20 to about 100 microns. In another embodiment, the average particle size of each component ranges from about 30 to about 60 microns

Two embodiments of the sustained release drug delivery composition according to the present invention are set forth in Table A.

TABLE A

| | Weight Ranges of Each | Weight Ranges of Each |
|---|---|---|
| | Component in Embodiment 1 | Component in Embodiment 2 |
| | (%/mg by weight of 250 mg | (%/mg by weight of 250 mg |
| Materials | capsule or lozenge) | capsule or lozenge) |
| Drug (for example | 4 to 40%/10 to 100 mg | 4 to 40%/10 to 100 mg |
| theophylline, glipizide or | | |
| acetaminophen) | | |
| Water-soluble polymer | 30 to 50%/75 to 125 mg | 35 to 40%/87.5 to 100 mg |
| Povidone (PVP) (MW | 20 to 45%/50 to 112.5 mg | 30 to 40%/75 to 100 mg |
| 1,000,000) | | |
| Hydroxypropyl cellulose | 20 to 45%/50 to 112.5 mg | 30 to 40%/75 to 100 mg |
| (HPC) (MW 95,000) | | |
| Fatty Acid | 20 to 45%/50 to 112.5 mg | 30 to 40%/75 to 100 mg |
| Stearic Acid | 20 to 45%/50 to 112.5 mg | 30 to 40%/75 to 100 mg |
| Lauric Acid | 20 to 45%/50 to 112.5 mg | 30 to 40%/75 to 100 mg |
| Palmitic Acid | 20 to 45%/50 to 112.5 mg | 30 to 40%/75 to 100 mg |

The present invention is also directed to a process for preparing the sustained release drug delivery composition of the present invention. This process comprises first mixing at least one of the drugs described hereinabove, at least one of the water-soluble polymers described hereinabove, and at least one of the fatty acids described hereinabove together to form a mixture, and then heating the thusly formed mixture at a sufficiently high temperature to melt the fatty acid and form water-soluble polymer—fatty acid solid solution in which the drug is either trapped or dissolved.

The drug, water-soluble polymer, and fatty acid can be mixed together with, for example, a tumble mixer or other conventional mixing apparatus. A person of ordinary skill in the art is readily familiar with the various conventional mixers that can be used in accordance with the present process.

The mixture of screened drug, screened water-soluble polymer and screened fatty acid is then heated to create a substantially uniform gel-like structure or matrix formed of a solid solution of the water-soluble polymer and fatty acid. The drug is either physically trapped, or dissolved in the gel-like structure or matrix and is later slowly released therefrom in a dissolution media, e.g. gastric acid and/or intestinal fluid, in a controlled manner either by diffusing through the matrix, or upon erosion of the matrix.

The mixture should be heated at a temperature sufficiently high to melt the fatty acid and form the solid solution in a reasonably short period of time without affecting the physical integrity of the selected dosage form, e.g. gelatin capsule shells. A person of ordinary kill in the art is generally capable of weighing the various factors and selecting an appropriate temperature. In one embodiment, the mixture is heated at a temperature ranging from about 30 to about 60° C. In another embodiment, the mixture is heated at a temperature ranging from about 35 to about 55° C. In yet another embodiment, the mixture is heated at a temperature ranging from about 40 to about 50° C.

The length of time the mixture should be heated generally depends on, for example, the type of water-soluble polymer being used, the molecular weight of the water-soluble polymer, the fatty acid being used, the heating temperature being used, and the drug load. A person of ordinary skill in the art, however, is generally capable of weighing the various factors and selecting an appropriate time period. In one embodiment, the mixture is heated for a period of time ranging from about 0.25 to about 10 hours. In another embodiment the mixture is heated for a period of time ranging from about 2 to about 6 hours.

The mixture can be heated with any conventional heating apparatus known to a person of ordinary skill in the art including, but not limited to, for example, a convection oven, water and steam bath.

In accordance with the process of the present invention, each of the water-soluble polymer, fatty acid, and drug can be separately screened (reduces average particle size) prior to being mixed together. In one embodiment, each screened component has an average particle size ranging about 20 to about 100 microns. In another embodiment, each screened component has an average particle size ranging from about 30 to about 60 microns. As the average particle size of each component can affect the sustained release properties of the final composition, screening each component can help insure consistently reproducible dissolution properties for the sustained release composition produced therefrom.

A person of ordinary skill in the art is familiar with typical screening devices that may be used including, but not limited to, for example, a mesh screen and any other method capable of producing the desired particle size.

If a sustained release capsule is being made in accordance with the process of the present invention, the water-soluble polymer, fatty acid, and drug mixture can be loaded into a capsule, and the loaded capsule subsequently heated in accordance with the process as more fully described hereinabove.

If a sustained release lozenge is being made in accordance with the process of the present invention, the water-soluble polymer, fatty acid, and drug mixture can be transferred to a lozenge mold, and the lozenge mold subsequently heated in accordance with the process as more fully described hereinabove.

In one embodiment of the present process, each of the drug, water soluble polymer, and fatty acid are screened so that each component has an average particle size of from about 20 to about 100 microns. The screened components are subsequently mixed together and the mixture transferred to a capsule or lozenge mold that is exposed to a temperature ranging from about 35° C. to about 55° C. for a period of time ranging from about 0.25 to about 10 hours.

### EXAMPLES

The present invention is further defined in the following Examples. It should be understood that these Examples are given by way of illustration only. From the above discussion and this Example, one skilled in the art can ascertain the essential characteristics of this invention, and without departing from the spirit and scope thereof, can make various changes and modifications to the invention to adapt the invention to various uses and conditions. As a result, the present invention is not limited by the illustrative examples set forth hereinbelow, but rather defined by the claims hereinbelow.

### Examples 1 and 2

Sustained release capsules containing theophylline having the following composition were prepared as described below:

| | Example No. | |
|---|---|---|
| | Example 1 capsule | Example 2 capsule |
| Drug | Theophylline (34 mg) | Theophylline (50 mg) |
| Water-Soluble | Hydroxypropylcellulose (HPC) | Povidone (PVP) |
| Polymer | (68 mg) | (MW 1,000,000) |
| | | (100 mg) |
| Fatty Acid | Lauric Acid (68 mg) | Stearic Acid (100 mg) |

Drug, water-soluble polymer and fatty acid were screened separately through a #40 mesh screen to produce particles of an average size of about 40 microns. The screened polymer and fatty acid in a 1:1 ratio together with screened drug were mixed in a tumble mixer for 5 minutes. The blend was then hand filled into size #0 white opaque capsules. The capsules were exposed to 50° C. in a convection oven for 2 hours to form sustained release theophylline capsules.

Drug release rate was tested for the Example 1 and Example 2 capsules. Dissolution of the theophylline capsules was conducted in 1000 mL of pH 6.6 phosphate buffer at 37° C. using the USP apparatus I at 100 rpm. The amount of theophylline dissolved was measured using a U.V. spectrophotometer at a wavelength of 270 nm.

Referring to Table 1 set out below, Example 2 capsules containing 50 mg theophylline, 100 mg PVP and 100 mg stearic acid released about 95% of the theophylline in about 7 hours.

Example 1 capsules containing 34 mg theophylline, 68 mg lauric acid and 68 mg HPC released about 90% of the theophylline over about 12 hours.

TABLE 1
Comparison of dissolution data of 34 mg or 50 mg theophylline capsules containing fatty acid and/or polymer (n = 6)

| | % theophylline dissolved (S.D.) at | | | | | | | | | | | | | | | |
|---|---|---|---|---|---|---|---|---|---|---|---|---|---|---|---|---|
| Formulations | 0.25 hr | 0.5 hr | 1 hr | 2 hr | 3 hr | 4 hr | 4.5 hr | 5 hr | 6 hr | 7 hr | 8 hr | 9 hr | 10 hr | 11 hr | 12 hr | 15 hr |
| Example 2 | 10 | 21 | 37 | 56 | 68 | 77 | 81 | 85 | 90 | 94 | 97 | 100 | 101 | | | |
| Theophylline | (4) | (6) | (7) | (6) | (6) | (5) | (5) | (4) | (3) | (2) | (2) | (1) | (1) | | | |
| (20%) + PVP90 | | | | | | | | | | | | | | | | |
| (40%) + stearic | | | | | | | | | | | | | | | | |
| acid (40%) | | | | | | | | | | | | | | | | |
| (50 mg | | | | | | | | | | | | | | | | |
| theophylline) | | | | | | | | | | | | | | | | |
| Example 1 | 8 | 14 | 23 | 36 | 44 | 53 | 57 | 61 | 66 | 71 | 76 | 81 | 85 | 89 | 92 | 100 |
| Theophylline | (1) | (2) | (2) | (2) | (1) | (1) | (0) | (0) | (1) | (1) | (2) | (2) | (2) | (2) | (2) | (2) |
| (20%) + lauric | | | | | | | | | | | | | | | | |
| acid (40%) + | | | | | | | | | | | | | | | | |
| HPC (40%) | | | | | | | | | | | | | | | | |
| (34 mg | | | | | | | | | | | | | | | | |
| theophylline) | | | | | | | | | | | | | | | | |

### Examples 3 and 4

Sustained release lozenges containing theophylline having the following composition were prepared as described below.

| | Example No. | |
|---|---|---|
| | Example 3 lozenge | Example 4 lozenge |
| Drug | Theophylline (34 mg) | Theophylline (50 mg) |
| Water-Soluble | Hydroxypropylcellulose (68 mg) | Povidone (100 mg) |
| Polymer | | |
| Fatty Acid | Lauric Acid (68 mg) | Stearic Acid (100 mg) |

Drug, water-soluble polymer and fatty acid were screened separately through a #40 mesh screen. The screened polymer and fatty acid together with screened drug were mixed in a tumble mixer for 5 minutes. The blend was then transferred to a lozenges mold. The mold was exposed to 50° C. in a convection oven for 2 hours. The resulting sustained release lozenges were removed from the mold to provide sustained release lozenges in accordance with the invention.

### Example 5

Sustained release capsules containing glipizide having the following composition were prepared as described below.

### Example 5 Glipizide Capsules

Glipizide 10 mg

Povidone (PVP 90) (80 mg)

Lauric Acid (80 mg)

Drug, povidone and lauric acid were screened separately through a #40 mesh screen. The screened polymer and fatty acid in a 1:1 ratio together with screened glipizide were mixed in a tumble mixer for 5 minutes. The blend was then hand filled into size #0 white opaque capsules. The capsules were exposed to 50° C. in a convection oven for 2 hours to form sustained release glipizide capsules.

Drug release rate was tested for the Example 5 glipizide capsules. Dissolution of the glipizide capsules was conducted in 1000 mL of simulated intestine fluid (SIF), pH 6.8, at 37° C. using the USP apparatus I at 100 rpm. The amount of glipizide dissolved was measured using a UV spectrophotometer at a wavelength of 276 nm.

### Example 6

### Comparison of Dissolution Profile of the Theo-24 and Glucotrol XL Commercial Products Versus Those Made Using the Method of the Invention

The dissolution of theophylline (Example 1) and glipizide (Example 5) capsules prepared using the method of the invention were compared against commercially available extended release dosage forms of each of these drugs, namely Theo-24 and Glucotrol XL, respectively. Theo-24 capsules are filled with coated beadlets and Glucotrol XL tablets are based on an osmotic drug delivery system. Even though the formulations prepared using the method of the invention were not optimized for a specific dissolution profile, the dissolution profiles obtained for theophylline and glipizide were very similar to commercially available extended release products (Tables 2 and 3).

TABLE 2
Dissolution comparison of 10 mg glipizide capsules (Example 5) with 10 mg
commercially available extended release 10 mg Glucotrol XL (n = 3)

| | % glipizide dissolved (S.D) at | | | | | | | | | | | | | | |
|---|---|---|---|---|---|---|---|---|---|---|---|---|---|---|---|
| Formulations | 0.25 hr | 0.5 hr | 1 hr | 2 hr | 3 hr | 4 hr | 4.5 hr | 5 hr | 6 hr | 7 hr | 8 hr | 9 hr | 10 hr | 11 hr | 12 hr |
| Example 5 | —¹ | 21 | 22 | 27 | 33 | 40 | 44 | 47 | 54 | 60 | 68 | 74 | 80 | 85 | 91 |
| Glipizide (5.88%) + | | (2) | (1) | (1) | (2) | (4) | (5) | (6) | (7) | (9) | (9) | (10) | (11) | (12) | (15) |
| PVP 90 (47.12%) + | | | | | | | | | | | | | | | |
| lauric acid (47.12%) | | | | | | | | | | | | | | | |
| Glucotrol XL | 11 | 17 | 16 | 15 | 17 | 23.3 | 28 | 33.2 | 41 | 49 | 58 | 67 | 77 | 87 | 98 |
| 10 mg | (1) | (3) | (4) | (4) | (3) | (3) | (3) | (4) | (4) | (5) | (7) | (8) | (10) | (11) | (13) |

¹“—” means the dissolution was not determined at that point in time.

TABLE 3
Dissolution comparison of 34 mg theophylline capsules (Example 1) with Theo-24,
commercially available extended release 100 mg theophylline capsules (n = 3)

| | % theophylline dissolved (S.D.) at | | | | | | | | | | | | | | | |
|---|---|---|---|---|---|---|---|---|---|---|---|---|---|---|---|---|
| Formulations | 0.25 hr | 0.5 hr | 1 hr | 2 hr | 3 hr | 4 hr | 4.5 hr | 5 hr | 6 hr | 7 hr | 8 hr | 9 hr | 10 hr | 11 hr | 12 hr | 15 hr |
| Example 1 | 8 | 14 | 23 | 36 | 44 | 53 | 57 | 61 | 66 | 71 | 76 | 81 | 85 | 89 | 92 | 100 |
| Theophylline 20% + | (1) | (2) | (2) | (2) | (1) | (1) | (0) | (0) | (1) | (1) | (2) | (2) | (2) | (2) | (2) | (2) |
| HPC 40% + | | | | | | | | | | | | | | | | |
| lauric acid 40% | | | | | | | | | | | | | | | | |
| Theo-24 | 1 | 4 | 9 | 19 | 30 | 40 | 45 | 50 | 58 | 65 | 72 | 78 | 82 | 86 | 90 | 97 |
| | (1) | (1) | (1) | (1) | (1) | (1) | (1) | (1) | (1) | (1) | (0) | (0) | (1) | (1) | (1) | (1) |

### Example 7

Sustained release capsules containing acetaminophen having the following composition were prepared as described below.

### Example 7 Acetaminophen

Acetaminophen (50 mg)

Povidone (PVP 90) (100 mg)

Stearic Acid (100 mg)

Drug, povidone and stearic acid were screened separately through a #40 mesh screen. The screened polymer and fatty acid in a 1:1 ratio together with screened acetaminophen were mixed in a tumble mixer for 5 minutes. The blend was then hand filled into size #0 white opaque capsules. The capsules were exposed to 50° C. in a convection oven for 2 hours to form sustained release acetaminophen capsules.

Drug release rate was tested for the Example 7 acetaminophen capsules. Dissolution of the acetaminophen capsules was conducted in 1000 mL of water, at pH 7.2, at 37° C. using the USP apparatus I at 100 rpm. The amount of acetaminophen dissolved was measured using a UV spectrophotometer at a wavelength of 249 nm.

### Examples 8 and 9

### Modifying Release by Changing the Drug Loading

The drug release rate was increased by increasing the drug loading in the formulation of the invention especially for a water-soluble drug like theophylline (Example 8) (prepared as described in Example 1). As shown in Table 4, after 4.5 hours, 60%, 68%, and 80% theophylline was dissolved for the formulations containing lauric acid and HPC (1:1) with 20%, 30%, and 40% drug loading, respectively. Similarly, for acetaminophen (Example 9) (prepared as described in Example 7), various dissolution profiles were obtained by changing the drug loading in PVP 90/stearic acid or HPC/lauric acid combinations (Table 5).

TABLE 4
Increase in dissolution of theophylline capsules with an increase in drug loading (n = 6)

| Formulations | % theophylline dissolved (S.D.) at | | | | | | | | | | | | | |
|---|---|---|---|---|---|---|---|---|---|---|---|---|---|---|
| Example 8 | 0.25 hr | 0.5 hr | 1 hr | 2 hr | 3 hr | 4 hr | 4.5 hr | 5 hr | 6 hr | 7 hr | 8 hr | 9 hr | 10 hr | 11 hr |
| Theophylline (20%) + | 9 | 17 | 25 | 40 | 48 | 56 | 60 | 64 | 69 | 74 | 79 | 84 | 87 | 91 |
| lauric acid (40%) + | (0) | (1) | (1) | (1) | (1) | (1) | (1) | (1) | (1) | (0) | (1) | (1) | (1) | (2) |
| HPC (40%) | | | | | | | | | | | | | | |
| (34 mg theophylline) | | | | | | | | | | | | | | |
| Theophylline (30%) + | 12 | 21 | 32 | 46 | 58 | 65 | 68 | 72 | 77 | 82 | 86 | 89 | 93 | 95 |
| lauric acid (35%) + | (2) | (2) | (2) | (3) | (4) | (5) | (5) | (5) | (6) | (6) | (7) | (7) | (7) | (6) |
| HPC (35%) | | | | | | | | | | | | | | |
| (51 mg theophylline) | | | | | | | | | | | | | | |
| Theophylline (40%) + | 16 | 27 | 38 | 54 | 65 | 74 | 80 | 83 | 90 | 95 | 98 | 99 | | |
| lauric acid (30%) + | (2) | (3) | (3) | (4) | (6) | (7) | (7) | (7) | (6) | (4) | (4) | (4) | | |
| HPC (30%) | | | | | | | | | | | | | | |
| (68 mg theophylline) | | | | | | | | | | | | | | |

TABLE 5
Increase in dissolution of acetaminophen capsules with an increase in drug loading (n = 6).

| Formulations | % acetaminophen dissolved (S.D.) at | | | | | | | | | | | | | | | |
|---|---|---|---|---|---|---|---|---|---|---|---|---|---|---|---|---|
| Example 9 | 0.25 hr | 0.5 hr | 1 hr | 2 hr | 3 hr | 4 hr | 4.5 hr | 5 hr | 6 hr | 7 hr | 8 hr | 9 hr | 10 hr | 11 hr | 12 hr | 15 hr |
| Acetaminophen (20%) + PVP | 3 | 9 | 16 | 22 | 25 | 29 | 31 | 32 | 35 | 37 | 39 | 41 | 43 | 45 | | |
| 90 (40%) + stearic | (1) | (1) | (1) | (2) | (2) | (2) | (3) | (3) | (3) | (3) | (4) | (4) | (4) | (4) | | |
| acid (40%) | | | | | | | | | | | | | | | | |
| (50 mg acetaminophen) | | | | | | | | | | | | | | | | |
| Acetaminophen (40%) + PVP | 16 | 29 | 42 | 56 | 64 | 70 | 72 | 73 | 76 | 77 | 79 | 80 | 80 | 81 | 82 | 84 |
| 90 (30%) + stearic | (7) | (9) | (10) | (10) | (10) | (10) | (10) | (10) | (10) | (9) | (9) | (9) | (9) | (9) | (8) | (8) |
| acid (30%) | | | | | | | | | | | | | | | | |
| (100 mg acetaminophen) | | | | | | | | | | | | | | | | |
| Acetaminophen (20%) + HPC | 14 | 27 | 41 | 57 | 67 | 75 | 78 | 80 | 84 | 87 | 89 | 90 | 91 | 92 | 92 | 93 |
| (40%) + lauric | (6) | (8) | (9) | (11) | (12) | (13) | (13) | (14) | (13) | (13) | (12) | (12) | (11) | (11) | (11) | (11) |
| acid (40%) | | | | | | | | | | | | | | | | |
| (34 mg acetaminophen) | | | | | | | | | | | | | | | | |
| Acetaminophen (40%) + HPC | 11 | 23 | 35 | 5 | 67 | 76 | 80 | 83 | 87 | 92 | 94 | 98 | 100 | 101 | 102 | |
| (30%) + lauric | (1) | (1) | (1) | (2) | (2) | (3) | (2) | (2) | (2) | (3) | (3) | (3) | (3) | (2) | (2) | |
| acid (30%) | | | | | | | | | | | | | | | | |
| (68 mg acetaminophen) | | | | | | | | | | | | | | | | |

### Examples 10 and 11

### Dissolution Stability of the Formulations

For any drug delivery system to be viable, the rate of drug release or the rate of dissolution should not change upon storage. For this purpose, theophylline capsules in HPC and lauric acid formulation or in PVP 90 and stearic acid formulation (Examples 10 and 11) were exposed to various conditions as listed in Tables 6 and 7, respectively. As shown in these Tables, the dissolution rate remained unchanged even after two weeks storage under accelerated conditions such as in open petri dishes at 30° C./60% RH or in closed HDPE bottles at 40° C./75% RH (Tables 6 and 7).

TABLE 6
Dissolution stability of 34 mg potency theophylline capsules (in HPC and lauric acid matrix)
after two weeks storage at various conditions (n = 6)

| Formulations | % theophylline dissolved (S.D.) at | | | | | | | | | | | | | | | |
|---|---|---|---|---|---|---|---|---|---|---|---|---|---|---|---|---|
| Example 10 | 0.25 hr | 0.5 hr | 1 hr | 2 hr | 3 hr | 4 hr | 4.5 hr | 5 hr | 6 hr | 7 hr | 8 hr | 9 hr | 10 hr | 11 hr | 12 hr | 15 hr |
| Theophylline | 12 | 21 | 30 | 44 | 54 | 62 | 66 | 69 | 74 | 78 | 82 | 86 | 89 | 92 | 95 | 103 |
| (20%) + HPC | (1) | (0) | (1) | (2) | (4) | (4) | (4) | (4) | (4) | (4) | (3) | (3) | (3) | (2) | (2) | (1) |
| (40%) + lauric | | | | | | | | | | | | | | | | |
| acid (40%) | | | | | | | | | | | | | | | | |
| Initial | | | | | | | | | | | | | | | | |
| at 30° C./ | 12 | 20 | 29 | 43 | 54 | 62 | 66 | 70 | 75 | 80 | 84 | 89 | 92 | 95 | 98 | 106 |
| 60% RH | (2) | (2) | (2) | (3) | (3) | (4) | (4) | (4) | (3) | (3) | (3) | (3) | (3) | (3) | (2) | (2) |
| in open Petri dish at | 12 | 20 | 28 | 40 | 49 | 57 | 60 | 63 | 68 | 73 | 77 | 80 | 84 | 87 | 90 | 99 |
| 30° C./60% RH | (2) | (2) | (3) | (3) | (4) | (4) | (3) | (3) | (3) | (3) | (2) | (2) | (2) | (2) | (2) | (3) |
| in HDPE at 40° C./ | 14 | 23 | 31 | 43 | 51 | 58 | 61 | 64 | 68 | 72 | 75 | 79 | 82 | 84 | 87 | 94 |
| 75% RH | (2) | (3) | (4) | (5) | (6) | (6) | (6) | (6) | (6) | (6) | (6) | (6) | (5) | (5) | (5) | (4) |

TABLE 7
Dissolution stability of 50 mg potency theophylline capsules (in PVP 90 and stearic acid matrix)
after two weeks storage at various conditions (n = 6)

| Formulations | % theophylline dissolved (S.D.) at | | | | | | | | | | | | | |
|---|---|---|---|---|---|---|---|---|---|---|---|---|---|---|
| Example 11 | 0.25 hr | 0.5 hr | 1 hr | 2 hr | 3 hr | 4 hr | 4.5 hr | 5 hr | 6 hr | 7 hr | 8 hr | 9 hr | 10 hr | 11 hr |
| Theophylline (20%) + PVP 90 | 2 | 11 | 26 | 46 | 57 | 67 | 72 | 76 | 81 | 87 | 91 | 96 | 100 | 104 |
| (40%) + stearic acid (40%) | (1) | (2) | (3) | (3) | (3) | (3) | (3) | (3) | (4) | (3) | (3) | (3) | (3) | (3) |
| Initial | | | | | | | | | | | | | | |
| in HDPE at 30° C./60% RH | 3 | 10 | 19 | 36 | 48 | 58 | 65 | 70 | 76 | 85 | 92 | 98 | 103 | 106 |
| | (1) | (2) | (3) | (3) | (3) | (3) | (2) | (2) | (2) | (3) | (4) | (4) | (3) | (3) |
| in open Petri dish at 30° C./ | 3 | 10 | 20 | 40 | 51 | 62 | 69 | 73 | 79 | 86 | 91 | 96 | 100 | 102 |
| 60% RH | (1) | (1) | (2) | (3) | (4) | (5) | (5) | (5) | (5) | (4) | (4) | (3) | (3) | (3) |
| in HDPE at 40° C./75% RH | 4 | 10 | 20 | 35 | 44 | 55 | 63 | 68 | 74 | 81 | 85 | 89 | 90 | 91 |
| | (1) | (2) | (1) | (2) | (2) | (3) | (3) | (4) | (5) | (5) | (5) | (4) | (5) | (6) |

What is claimed is:

1. A pharmaceutical delivery composition in the form of a gel-like structure comprising:
a) a water-soluble polymer; and
b) a fatty acid,
whereby said composition delivers a drug in a sustained release manner.

2. The composition as defined in claim 1, wherein the water-soluble polymer and the fatty acid form a solid solution.

3. The composition as defined in claim 2, wherein the solid solution is formed by mixing the water-soluble polymer and the fatty acid together, and then exposing the mixture to a temperature ranging from about 30 to about 60° C.

4. The composition as defined in claim 1, wherein the water soluble polymer and the fatty acid each separately have an average particle size of from about 20 to about 100 microns.

5. The composition as defined in claim 1, wherein the water-soluble polymer and the fatty acid are present in a weight ratio of water-soluble polymer to fatty acid ranging from about 2.5:1 to about 1:2.

6. The composition as defined in claim 1, wherein the water-soluble polymer is povidone, hydroxypropyl cellulose, hydroxypropyl methylcellulose, or polyethylene glycol.

7. The composition as defined in claim 1, wherein the fatty acid is stearic acid, palmitic acid, or lauric acid.

8. The composition as defined in claim 1, wherein the water-soluble polymer is povidone or hydroxypropyl cellulose, the fatty acid is stearic acid or lauric acid, and the water-soluble polymer and the fatty acid are present in a weight ratio of water-soluble polymer to fatty acid of about 1:1.

9. A sustained release drug delivery composition comprising:
a) at least one drug; and
b) a gel-like structure comprising:
1) at least one water-soluble polymer; and
2) at least one fatty acid,
wherein the drug is trapped or dissolved in the gel-like structure, and the drug is released from the gel-like structure into a dissolution medium at a sustained release rate.

10. The composition as defined in claim 9, wherein the water-soluble polymer and the fatty acid form a solid solution and said drug is trapped in said solid solution.

11. The composition of claim 9, wherein the water-soluble polymer, the fatty acid, and the drug form a solid solution.

12. The composition as defined in claim 10, where said solid solution is formed by exposing the mixture to a temperature ranging from at least about 30° C. to about 60° C.

13. The composition as defined in claim 9, wherein the water-soluble polymer and the fatty acid are present in a water-soluble polymer to fatty acid weight ratio ranging from about 2.5:1 to about 1:2.

14. The composition as defined in Claim9, wherein the water-soluble polymer, the fatty acid, and the drug have an average particle size of from about 20 to about 100 microns.

15. The composition as defined in claim 9, wherein the water-soluble polymer is povidone, hydroxypropyl cellulose, hydroxypropyl methylcellulose, or polyethylene glycol.

16. The composition as defined in claim 9, wherein the fatty acid is stearic acid, palmitic acid, or lauric acid.

17. The composition as defined in claim 9, wherein the water-soluble polymer is povidone or hydroxypropyl cellulose, the fatty acid is stearic acid or lauric acid, and the water-soluble polymer and the fatty acid are present in a weight ratio of water-soluble polymer to fatty acid of about 1:1.

18. The composition as defined in claim 9, wherein said composition is in a form selected from a capsule and a lozenge.

19. The composition as defined in claim 9, wherein the drug is a pharmacologically active substance.

20. The composition as defined in claim 19, wherein the pharmacologically active substance is theophylline, acetaminophen, or glipizide.

21. The composition as defined in claim 9, wherein the composition comprises from about 20 to about 50%, by weight composition, of the at least one water-soluble polymer; from about 20 to about 50%, by weight composition, of the at least one fatty acid; and from about 3 to about 60%, by weight composition, of the at least one drug.

22. A method for preparing the sustained release drug delivery composition of claim 9, comprising:
a) mixing the drug, the water-soluble polymer, and the fatty acid together;
and
b) heating the mixture of a) at a sufficiently high temperature to melt the fatty acid, wherein said water-soluble polymer and said fatty acid form a solid solution and said drug is trapped or dissolved in said solid solution

23. The method as defined in claim 22, further comprising separately screening each of the drug, the water-soluble polymer, and the fatty acid prior to mixing said drug, said water-soluble polymer, and said fatty acid together in step a), wherein said screening results in each of the drug, the water-soluble polymer, and the fatty acid having an average particle size of from about 20 to about 100 microns.

24. The method as defined in claim 22, wherein the temperature at which the mixture of a) is heated in b) ranges from about 30 to about 60° C., said mixture being heated in b) for a period of time ranging from about 0.25 to about 10 hours.

25. The method as defined in claim 23, further comprising loading a capsule with the mixture of a), and then subsequently heating the loaded capsule in accordance with step b).

26. The method as defined in claim 23, further comprising transferring the mixture of a) to a lozenge mold, and then subsequently heating said mixture in accordance with step b).

27. A method for preparing the sustained release drug delivery composition of claim 9, comprising:
a) screening each of the at least one drug, the at least one water-soluble polymer, and the at least one fatty acid so that said drug, said water-soluble polymer, and said fatty acid each have an average particle size of from about 20 to about 100 microns;
b) mixing the drug, the water-soluble polymer, and the fatty acid together;
c) transferring the mixture of b) to a capsule or a lozenge mold; and
d) heating the mixture of c) to a temperature ranging from about 35 to about 55° C. for a period of time ranging from about 0.25 to about 10 hours.
